(19) United States
(12) Patent Application Publication
(10) Pub. No.: US 2011/0135735 A1
(43) Pub. Date: Jun. 9, 2011

(54) PROCESS FOR PRODUCTION OF A COMPOSITE MATERIAL HAVING ANTIMICROBIAL ACTIVITY

(75) Inventor: Peter Steinruecke, Nuernberg (DE)

(73) Assignee: BIO-GATE AG, Nuernberg (DE)

(21) Appl. No.: 12/737,266

(22) PCT Filed: Jun. 25, 2009
(86) PCT No.: PCT/EP2009/057992

§ 371 Date: Feb. 14, 2011

(30) Foreign Application Priority Data
Jul. 15, 2008 (DE) 10 2008 033 224.0

(51) Int. Cl. A61K 9/14; B05D 5/00; A61K 33/24; A61K 33/38; A61K 33/34; A61K 33/30; A61P 31/00

(52) U.S. Cl. 424/489; 427/216; 424/649; 424/618; 424/646; 424/617; 424/650; 424/638; 424/651; 424/641

(57) ABSTRACT

The invention relates to a process for production of a composite material having antimicrobial activity, having the following steps: provision of a metal powder produced from a metal having antimicrobial activity, wherein the metal powder is formed from discrete agglomerates having a porosity of 30 to 98%, wherein the agglomerates have a spongy structure formed by solid material bridges; melting a thermoplastic and setting a predetermined viscosity; mixing the metal powder with the molten thermoplastic in a predetermined quantitative ratio; and cooling the mixture, wherein the metal powder is firmly bound to a matrix formed by the plastic.

The invention relates to a process for production of a composite material having antimicrobial activity.

U.S. Pat. No. 5,837,275 discloses an antimicrobial coating wherein nano particles made of silver are applied via sputtering to a surface to be coated. Powders made of nano particles have the disadvantageous property that it is extremely difficult to disperse them homogeneously in a liquid or a resin. Apart from this, nano particles tend to create relatively hard agglomerate. This also counteracts a homogeneous distribution of the nano particles in a composite material.

WO 02/17984 A1 describes an antimicrobial material for implantation in bones. To create the material, porous silver aggregates are first stirred into a synthetic resin and completely infiltrated with the synthetic resin. The synthetic resin is then hardened. During the making of the known composite material, the problem occurs that the silver aggregates following the force of gravity always tend to accumulate on the bottom of the container provided to hold the synthetic resin. Although this can be counteracted by increasing the viscosity of the synthetic resin, in this case however, the problem occurs that the silver aggregates are not completely infiltrated. This in turn reduces the antimicrobial effectiveness of the composite material.

It is an object of this invention to eliminate the problems as per prior art. In particular, a process for making a composite material with antimicrobial activity is to be specified which can be carried out simply and inexpensively. A further goal of the invention is to specify a composite material with improved antimicrobial effectiveness which can be made as simply as possible.

This object is resolved by the features of claims 1, 2 and 19. Useful embodiments of the invention result from the features of claims 3 to 18 and 20 to 28.

In accordance with a first aspect of the invention, a process for the making of a composite material with antimicrobial activity is provided with the following steps:
Provision of a metal powder made of an antimicrobial-acting metal, wherein the metal powder is created from discrete agglomerates having a porosity of 30 to 98%, wherein the agglomerates have a spongy framework structure created by solid material bridges;
Melting on a thermoplastic synthetic material and setting a specified viscosity;
Mixing the metal powder with the melted on thermoplastic synthetic material in a specified proportion; and
Cooling off the mixture, wherein the metal powder is firmly connected with a matrix created by the synthetic material.

The agglomerates provided by the invention have a firm spongy framework structure. The spongy framework structure surrounds an open pore volume. An open porosity in the sense of this invention is defined by

θ=(1−ρ/ρ₀)*100%

wherein
ρ is the gross density of the metal and
ρ₀ is the true density of the metal.

The agglomerates provided by the invention have the advantage that their framework structure is not destroyed when it is incorporated into a thermoplastic melted mass. This means that the porosity of the agglomerates is retained. From the agglomerates provided by the invention, aggregates are to be distinguished which are created by chance from nano particles and essentially not from solid material bridges but are connected with each other by attractive electrostatic forces. Such aggregates change their structure while being incorporated into a thermoplastic melted mass. In particular, in the incorporated state, they do not have the porosity which can be obtained by the agglomerates provided by the invention.

Using the agglomerates provided by the invention, a composite material with high antimicrobial effectiveness can be made in a surprisingly simple and inexpensive manner.

As provided by the invention, a metal powder is used whose particles are created from discrete porous agglomerates. This means that the proposed composite material is also particularly suitable for the making of implants, catheters and similar. The proposed agglomerates have no undesirable cyto-toxic effect. At the same time, they have a large inner surface which permits a release of a relatively high rate of metal ions causing an antimicrobial activity. By using a thermoplastic synthetic material as provided by the invention to make the composite material, a particularly uniform and homogeneous distribution of the metal powder can be achieved in the composite material.

Firstly a semi-finished product can be made with the proposed process. This can be a granulate, rods, plates or similar. In a further step of the process, the semi-finished product can be processed to a desired molded body.

According to a further aspect of the invention, a process is provided with the following steps for making a composite material having antimicrobial properties:
Provision of a metal powder made of an antimicrobial-acting metal, wherein the metal powder is created from discrete agglomerates having a porosity of 30 to 98%, wherein the agglomerates have a spongy structure created by solid material bridges;
Provision of a synthetic powder made from a thermoplastic synthetic material;
Mixing of the metal powder and the synthetic powder in a specified proportion;
Heating up a mixture created from the metal powder and the synthetic powder to a temperature in the range of the melting temperature of the synthetic powder; and
Cooling off the mixture, wherein the metal powder is firmly connected with a matrix created by the thermoplastic synthetic material.

In contrast to the above proposed process in accordance with the first aspect of the invention, in accordance with the second aspect of the invention, a mixture is first made from the metal powder and the synthetic powder. Such a mixture is easy to make. It can be intermediately stored as an intermediate product. Semi-finished products or shaped parts can be made from this. For this purpose, the mixture of the metal powder and the synthetic powder is heated to a temperature in the range of the melting temperature of the synthetic powder.

According to an embodiment of the process, a pressed body can be made via pressing from the mixture before the step of heating up the mixture. The pressed body can be a molded body which is then compressed by the heat and pressure treatment provided by the invention.

It has been shown to be useful that a medium grain size of the synthetic particles making up the synthetic powder corresponds approximately to a medium grain size of the agglomerates. This permits the making of a particularly homogeneous mixture.

The embodiments described below can be applied to both aspects of the process provided by the invention.

According to an advantageous embodiment, a pressure which is different from the surrounding pressure is applied to the mixture. This pressure can be a pressure that is greater than the surrounding pressure. This causes the melted mass of the thermoplastic synthetic material or the thermoplastic melted mass to be pressed into the open pore volume of the agglomerates. But this pressure can also be an underpressure. In other words, a pressure that is less than the surrounding pressure. Under the influence of the underpressure, the air escapes from inside the mixture, in particular from the pore volume of the agglomerates. This also supports the infiltration of the thermoplastic melted mass into the pore volume of the agglomerates. If an over- or underpressure is applied to the mixture, care must be taken that this is selected in such a manner that the spongy framework structure of the agglomerates is not destroyed. The amount of pressure to be applied depends on the structure of the agglomerates, the viscosity of the thermoplastic melted mass, the type and amount of additives and similar.

According to an embodiment, it is provided that the step of heating up and applying a pressure are performed at the same time. In other words, the mixture is advantageously pressed hot. With this, a particularly effective compression of the material can be achieved.

According to a particularly advantageous embodiment feature, the pressure can also be applied to the mixture with shaping via injection molding or extrusion. For this purpose, for example, the mixture can first be made in a compounder with axially movable screw. After the mixture is made, a pressure can then be applied to the mixture by an axial movement of the screw and thereby, the mixture can be extruded through a mouthpiece. An axial movement of the screw also makes it possible to shoot the mixture under pressure into an injection mold.

According to a further embodiment, it is also possible to evacuate the mixture during heating up and/or applying a pressure. This succeeds in making a particularly dense and almost pore-free composite material.

According to a further embodiment, the thermoplastic synthetic material is selected from the following group: acrylonitrile butadiene styrene (ABS), acrylic, celluloid, cellulose acetate, ethylene vinyl acetate (EVA), ethylene vinyl alcohol (EVAL), fluoroplasts (PTFE, FEP, PFA, CTFE, ECTFE, ETFE), ionomers, Kydex®, liquid crystal polymer (LCP), polyacetal (POM or acetal), polyacrylates (acrylic), polyacrylonitrile (PAN or acrylonitrile), polyamide (PA), polyamide imide (PAI), polyacrylic ether ketone (PAEK), polybutadiene (PBD), polybutylene (PB), polybutylene terephthalate (PBT), polycaprolacetone (PCL), polychlorotrifluoroethylene (PCTFE), polyethylene terephthalate (PET), polycyclohexylendimethylene terephthalate (PCT), polycarbonate (PC), polyhydroxyalkanoate (PHAs), polyketone (PK), polyester, polyethylene (PE), polyetheretherketone (PEEK), polyetherimide (PEI), polyethersulfone (PES), polyethylenchlorinate (PEC), polyimide (PI), polyactic acid (PLA), polymethylpenten (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC), Spectralon®. The composite material which is made particularly using the previously stated thermoplastic synthetic materials has many uses due to its antimicrobial activity. It is particularly suitable as material for making refrigerators, drug delivery systems, mechanical shock absorbers in shoes, insulating material, blood vessel implants, functional textiles, technical textiles, hoses, cables, laminates and windows, membranes, seals, instrument consoles, door coverings, seat coverings, jalousies, trays, safety helmets, interior coverings of aircrafts, ventilation systems, implants, intraocular lenses, artificial teeth, tooth fillings, adhesives, artificial fingernails, super absorbers, bladder catheters, suture material, textile fibers, catheter tubes, components for dialysis devices, syringes, heart valves, carpet fibers, fishing lines, pantyhoses, bristles for tooth brushes, re-absorbable suture material, artificial blood vessels, tendon and ligament replacement, packaging material, surgical anchoring materials such as screws, bone plates, bone plate systems, surgical nets, cardiovascular patches, stents, tissue repair devices, meniscal augmentation devices, skin substitute materials, bone substitute materials, wound dressings, nerve substitute materials, sockets for artificial hip joints, artificial knee joints, hip joints, for the making of ultrasonic heads, components for blood oxygenators and kidney dialysis, artificial finger joints, extra corporal blood tubes, blood bags, bags for intravenous applications, and similar.

Regarding a particularly efficient antimicrobial activity, it has been shown to be useful to use agglomerates whose medium grain size is in the range from 1 to 30 μm, preferably in the range from 5 to 25 μm. Agglomerates with the proposed medium grain size can be dispersed well in a thermoplastic melted mass. A homogeneous composite material can be made with this.

The agglomerates advantageously have a density in the range of 0.4 to 1.8 g/cm³. The density of the agglomerates is similar to the density of the thermoplastic synthetic material. This can be used advantageously to avoid decomposition of the metal powder due to gravity. The metal powder distributes itself uniformly in the mixture and consequently in the composite material made from that.

The agglomerates which are used, advantageously have a porosity of from 70 to 98% or from 80 to 95%. Thus their making only requires a relatively small amount of antimicrobial-acting metal.

According to an advantageous embodiment, the agglomerates are created from primary particles which are firmly connected with each other via sinter necks. In this connection, the primary particles have a medium grain size in the range from 10 to 100 nm. The metal powder or such agglomerates can be made via inert gas vaporization. The antimicrobial-acting metal advantageously contains one or more of the following elements as the main component: Ag, Au, Pt, Pd, Ir, Sn, Cu, Sb, Zn. The antimicrobial-acting metal preferably essentially contains Ag.

According to a further particularly advantageous embodiment, the agglomerates can be infiltrated with a fluid, a wax or a polymer before the step of making a mixture with the thermo-plastic synthetic material. Such infiltrated agglomerates are particularly pressure proof. In other words, they can be incorporated into a thermoplastic melted mass under a high pressure and, in particular, can also be processed via extrusion or using injection molding procedure. The proposed process step of infiltrating is used in particular then when the agglomerates are incorporated into a thermoplastic melted mass with a high viscosity or when, for process technology reasons, the mixture is to be exposed to a high pressure. The fluid, the wax or the polymer which is used for the infiltration of the agglomerates are selected in such a manner that the material properties of the thermoplastic synthetic material are not negatively affected. In particular, the infiltrated fluid or the infiltrated wax can be substances which are usually used as additives, for example, for the liquefaction of thermoplastic melted masses. The polymer is advantageously a substance which binds with the respective thermoplastic synthetic material being used or is dissolved therein. The fluid material used for the infiltration can also be colored. This makes it possible to change the appearance of a composite material containing the agglomerates.

Regarding the process technology, it has further been shown to be particularly useful to use a, preferably heatable, compounder to make the mixture. The compounder can have an axially movable screw. A twin-screw compounder can also be used.

A pressure of more than 0.5*10⁵ Pa, preferably more than 5*10⁵ Pa, is advantageously exerted on the mixture. The previously mentioned specification of the pressure is understood to mean “overpressure.” In other words, this is a pressure which is exerted on the mixture in addition to the surrounding air pressure. The pressure can be exerted mechanically or also with a gas which is under pressure. Advantageously, the pressure is exerted on the mixture for at least a duration of 0.1 to 120 seconds. The specified minimum holding time is required so that an essentially complete infiltration of the agglomerates with the thermoplastic synthetic material is ensured. The holding time depends essentially on the viscosity of the melted mass of the thermoplastic synthetic material. Longer holding times are possible.

According to a further aspect of the invention, a composite material with antimicrobial activity is provided for which discrete agglomerates having a porosity of 30 to 98% and being made of an antimicrobial-acting metal are held in a matrix created from a thermoplastic synthetic material wherein the agglomerates have a spongy framework structure created by solid material bridges.

The agglomerates can be created in particular from primary particles which are firmly connected with each other via sinter necks. In this connection, the primary particles can have a medium grain size in the range from 10 to 100 nm. The term “sinter neck” is understood to mean a material bridge between two adjacent primary particles. Sinter necks are created during the early phase of sintering by diffusion processes. Such “sinter necks” are described indeed in connection with the process of “sintering.” But it is also possible that sinter necks are formed by other processes during which similar conditions exist as with sintering.

But agglomerates with the spongy framework structure provided by the invention can also be made in other ways. For example, it is possible to foam up metal melted masses using foaming agents in a suitable manner. Moreover, it is possible to make an inhomogeneous mixture of a noble and a base metal and then dissolve the base metal selectively with acid treatment so that a spongy highly-porous framework structure created from the more noble metal will remain.

The agglomerates are advantageously contained in an amount of 0.1 to 5.0 percent by weight. The specified low amounts are already sufficient to give the composite material an antimicrobial activity.

Reference is made to the preceding explanations covering the further embodiment features of the composite material. The features described there can also be used correspondingly for the features of the composite material.

The process provided by the invention makes it possible for the first time to provide thermoplastic composite materials having a relatively high melting point with an antimicrobial activity in a relatively simple and inexpensive manner. Up to now, conventional antimicrobial-acting organic additives have not been able to be used to make composite materials having a high melting point due to their lack of sufficient temperature stability. In contrast, using the agglomerates provided by the invention makes it possible to provide even thermoplastic synthetic materials with an antimicrobial activity although they have high melting points.

Exemplary embodiments will now be used to describe the invention in more detail.

### EXEMPLARY EMBODIMENT 1

Polyoxyethylene (Hostaform C 9021 GV1/10) was melted at a temperature of 190° C. in a PolyDriveThermo Haake kneader (Haake company, Karlsruhe, Germany). The melted mass was then mixed with 0.5 percent by weight of metal powder at a speed of 70 revolutions/minute. The metal powder consisted of silver agglomerates with a porosity of approximately 80% and a medium grain size of approximately 25 μm. The medium grain size of the primary particles was 20 to 50 nm.

The mixture was stirred at 190° C. for approximately 8 minutes. Then the melted mass was shaped between two brass plates into flat disks and, after cooling off, processed in a granulator (type C13.20vs, of the Wanner Technik GmbH company) into a granulate with a medium diameter of approximately 3 mm.

To test the antimicrobial effectiveness of the granulate, 66.7 g of granulate was suspended in one liter of a diluted sodium nitrate solution (7 mM) and incubated at room temperature for a period of 72 hours. Afterwards, voltammetry was used to determine the concentration of the silver ions in the supernatant. It was found that the supernatant has a silver content of 2.1 μM per liter. The measured concentration of silver ions is antimicrobial-acting.

### EXEMPLARY EMBODIMENT 2

Polyurethane (Elastolan C85A10 of the BASF AG company) was melted at a temperature of 185° C. in a PolyDriveThermo Haake kneader (Haake company, Karlsruhe, Germany). The melted mass was then mixed with 0.5 percent by weight of the metal powder described in explanatory example 1. The melted mass mixed with the metal powder was stirred at 70 revolutions/minute for 8 minutes. Then the melted mass was shaped between two brass plates into flat disks. After cooling off, the flat disks were processed in a granulator (type C13.20vs, of the Wanner Technik GmbH company) into a granulate with a medium diameter of approximately 3 mm.

A measurement as described above of the concentration of the emitted silver ions resulted in a concentration of 1.6 μM silver ions per liter. Such a concentration of silver ions is antimicrobial-acting.

### EXEMPLARY EMBODIMENT 3

Polyacetal (PQM Delrin 500 NC010, of the Dupont company) was melted in an extruder at a temperature of 214° C. and mixed with 3 percent by weight of the above described silver powder. The melted mass was extruded with a throughput of 20 kg/hour at a speed of 370 revolutions/minute and an operating pressure of 24 bar. A granulate was made from the extruded material.

In turn, a measurement of the silver ions revealed that the material is antimicrobial-acting.

1. A process for production of a composite material having antimicrobial activity, with the following steps:
Provision of a metal powder made from an antimicrobial-acting metal, wherein the metal powder is created from discrete agglomerates having a porosity of 30 to 98%, wherein the agglomerates have a spongy framework structure created by solid material bridges;
Melting a thermoplastic synthetic material and setting a specified viscosity;
Mixing the metal powder with the melted thermoplastic synthetic material in a specified proportion; and
Cooling off the mixture, wherein the metal powder is firmly connected with a matrix created by the synthetic material.

2. A process for production of a composite material having antimicrobial properties, with the following steps:
Provision of a metal powder made from an antimicrobial-acting metal, wherein the metal powder is created from discrete agglomerates having a porosity of 30 to 98%, wherein the agglomerates have a spongy framework structure created by solid material bridges;
Provision of a synthetic powder made from a thermoplastic synthetic material;
Mixing of the metal powder and the synthetic powder in a specified proportion;
Heating up a mixture created from the metal powder and the synthetic powder to a temperature in the range of the melting temperature of the synthetic powder; and
Cooling off the mixture, wherein the metal powder is firmly connected with a matrix created by the thermoplastic synthetic material.

3. The process as defined in claim 2, wherein a pressed body is pressed out of the mixture before the step of heating up the mixture.

4. The process as defined in claim 2, wherein a medium grain diameter of the synthetic particles which create the synthetic powder corresponds approximately to a medium grain diameter of the agglomerates.

5. The process as defined in claim 1, wherein a pressure different from the surrounding pressure is exerted on the mixture.

6. The process as defined in claim 1, wherein the step of heating up and exerting the pressure are performed at the same time.

7. The process as defined in claim 1, wherein the pressure is applied to the mixture during shaping via injection molding or extrusion.

8. The process as defined in claim 1, wherein the thermoplastic synthetic material is selected from the following group: acrylonitrile butadiene styrene (ABS), acrylic, celluloid, cellulose acetate, ethylene vinyl acetate (EVA), ethylene vinyl alcohol (EVAL), fluoroplasts (PTFE, FEP, PFA, CTFE, ECTFE, ETFE), ionomers, Kydex®, liquid crystal polymer (LCP), polyacetal (POM or acetal), polyacrylates (acrylic), polyacrylonitrile (PAN or acrylonitrile), polyamide (PA), polyamide imide (PAI), polyacrylic ether ketone (PAEK), polybutadiene (PBD), polybutylene (PB), polybutylene terephthalate (PBT), polycaprolacetone (PCL), polychlorotrifluoroethylene (PCTFE), polyethylene terephthalate (PET), polycyclohexylendimethylen terephthalate (PCT), polycarbonate (PC), polyhydroxyalkanoate (PHAs), polyketone (PK), polyester, polyethylene (PE), polyetheretherketone (PEEK), polyetherimide (PEI), polyethersulfone (PES), polyethylenchlorinate (PEC), polyimide (PI), polyactic acid (PLA), polymethylpenten (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC), Spectralon®.

9. The process as defined in claim 1, wherein a medium grain size of the agglomerates is in the range from 1 to 30 μm, preferably in the range from 5 to 25 μM.

10. The process as defined in claim 1, wherein the agglomerates have a density in the range from 0.4 to 1.8 g/cm³.

11. The process as defined in claim 1, wherein the agglomerates are created from primary particles which are firmly connected with each other via sinter necks.

12. The process as defined in claim 1, wherein a medium grain size of the primary particles is in the range from 10 to 100 nm.

13. The process as defined in claim 1, wherein the metal powder is made via inert gas vaporization.

14. The process as defined in claim 1, wherein the antimicrobial-acting metal contains one or more of the following elements as the main component: Ag, Au, Pt, Pd, Ir, Sn, Cu, Sb, Zn.

15. The process as defined in claim 1, wherein the agglomerates are infiltrated with a fluid, a wax or a polymer before the step of making a mixture with the thermoplastic synthetic material.

16. The process as defined in claim 1, wherein a preferably heatable compounder is used to make the mixture.

17. The process as defined in claim 1, wherein a pressure of more than 0.5*10⁵ Pa, preferably more than 5*10⁵ Pa is exerted on the mixture.

18. The process as defined in claim 1, wherein the pressure is exerted on the mixture for a duration of at least 0.1 to 120 seconds.

19. A composite material having antimicrobial activity for which discrete agglomerates having a porosity of 30 to 98% and being made from an antimicrobial-acting metal are held in a matrix created from a thermoplastic synthetic material, wherein the agglomerates have a spongy framework structure created by solid material bridges.

20. The composite material as defined in claim 19, wherein the agglomerates are contained in an amount of 0.1 to 5.0 percent by weight.

21. The composite material as defined in claim 19, where the thermoplastic synthetic material is selected from the following group: acrylonitrile butadiene styrene (ABS), acrylic, celluloid, cellulose acetate, ethylene vinyl acetate (EVA), ethylene vinyl alcohol (EVAL), fluoroplasts (PTFE, FEP, PFA, CTFE, ECTFE, ETFE), ionomers, Kydex®, liquid crystal polymer (LCP), polyacetal (POM or acetal), polyacrylates (acrylic), polyacrylonitrile (PAN or acrylonitrile), polyamide (PA), polyamide imide (PAI), polyacrylic ether ketone (PAEK), polybutadiene (PBD), polybutylene (PB), polybutylene terephthalate (PBT), polycaprolacetone (PCL), polychlorotrifluoroethylene (PCTFE), polyethylene terephthalate (PET), polycyclohexylendimethylen terephthalate (PCT), polycarbonate (PC), polyhydroxyalkanoate (PHAs), polyketone (PK), polyester, polyethylene (PE), polyetheretherketone (PEEK), polyetherimide (PEI), polyethersulfone (PES), polyethylenchlorinate (PEC), polyimide (PI), polyactic acid (PLA), polymethylpenten (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC), Spectralon®.

22. The composite material as defined in claim 19, where a medium grain size of the agglomerates is in the range from 1 to 30 μm, preferably in the range from 5 to 25 μm.

23. The composite material as defined in claim 19, wherein the agglomerates have a density in the range from 0.4 to 1.8 g/cm³.

24. The composite material as defined in claim 19, wherein the agglomerates are created from primary particles which are firmly connected with each other via sinter necks.

25. The composite material as defined in claim 19, wherein a medium grain size of the primary particles is in the range from 10 to 100 nm.

26. The composite material as defined in claim 19, wherein the agglomerates are made via inert gas vaporization.

27. The composite material as defined in claim 19, wherein the antimicrobial-acting metal contains one or more of the following elements as the main component: Ag, Au, Pt, Pd, Ir, Sn, Cu, Sb, Zn.

28. The composite material as defined in claim 19, wherein the agglomerates are essentially completely infiltrated with the thermoplastic synthetic material, a fluid, a wax or a polymer.
